(19) United States
(12) Patent Application Publication
(10) Pub. No.: US 2020/0206291 A1
(43) Pub. Date: Jul. 2, 2020

(54) COMPOSITION FOR PREVENTING OR TREATING OBESITY, COMPRISING SUPPLEMENT FOR REDUCING BODY FAT AND B-GLUCAN AS ACTIVE INGREDIENTS

(71) Applicant: QUEGEN BIOTECH CO., LTD., Siheung-si (KR)

(72) Inventors: Jong Dae LEE, Seoul (KR); Je Phil RYOO, Gwangmyeong-si (KR); Seung In CHOI, Seoul (KR); Je Kyoung KIM, Bucheon-si (KR); Yong Man JANG, Siheung-si (KR); Min Ji KIM, Donghae-si (KR); Eun Ji PARK, Gwangju (KR); Chul Su YANG, Ansan-si (KR)

(73) Assignee: QUEGEN BIOTECH CO., LTD., Siheung-si (KR)

(21) Appl. No.: 16/609,734

(22) PCT Filed: Aug. 29, 2018
(86) PCT No.: PCT/KR2018/009976

§ 371 Date: Oct. 30, 2019

(30) Foreign Application Priority Data
Sep. 19, 2017 (KR) 10-2017-0120338
Aug. 28, 2018 (KR) 10-2018-0101389

(51) Int. Cl. A61K 36/38; A61K 31/716; A61K 31/201; A61P 3/04

(52) U.S. Cl. CPC A61K 36/38; A61P 3/04; A61K 31/201; A61K 31/716

(57) ABSTRACT

The present invention relates to a composition for preventing or treating obesity, comprising a supplement for reducing body fat and a β-glucan as active ingredients.

### FIELD OF THE DISCLOSURE

The present invention relates to a composition for preventing or treating obesity, which includes a supplement for reducing body fat and beta-glucan (β-glucan) (particularly, Schizophyllum commune-derived β-glucan) as active ingredients.

### DESCRIPTION OF RELATED ART

Obesity is a condition in which an excessive increase in body fat and subsequent metabolic abnormalities thereby are induced. Obesity is considered to be a major risk factor for various adult diseases such as hypertension, diabetes and cardiovascular diseases, and cancer. As in other countries, the prevalence of obesity among Korean adults has been increasing steadily over the last two decades. Some years ago, the World Health Organization (WHO) has defined obesity as a “new infectious disease in the 21^{st} century,” and the American Medical Association has defined obesity as a “disease.” Such global movement suggests that obesity is no longer a cosmetic and physical problem but a “disease” requiring treatment. Recently, in Korea, movements for resolving obesity, for example, governments' obesity campaigns as well as endeavors in the medical and pharmaceutical sectors are growing. Like the advanced medical countries, Korean people are raising the awareness of obesity and creating awareness to define obesity as a disease. Obesity has been reported to be associated with type 2 diabetes, cardiovascular diseases, osteoarthritis, some types of cancer, sleep apnea syndrome, asthma and non-alcoholic fatty liver. For anti-obesity drug treatment, in addition to weight loss, the alleviation of diseases associated with obesity, such as high blood pressure, dyslipidemia and atherosclerotic heart disease, is even more important.

Today, as a therapeutic method for obesity, there is chemotherapy (diet supplement). According to a survey conducted by a consumer group in 2006, it has been reported that more than half of Seoul's citizens take dietary supplements, which is because eating something is easier than maintaining a healthy lifestyle. To resolve obesity, it is easier to eat dietary supplements that seem to be more effective than a hard diet, increased physical activity and regular exercise. As such, diet supplements are essential for modern obesity treatments. Common dietary supplements generally include ingredients such as a Garcinia cambogia extract (e.g., hydroxycitric acid (HCA)), conjugated linoleic acid (CLA), and dietary fiber. Although there is a difference between supplements, they have been shown to be effective in suppressing appetite, fat accumulation, heat generation and reduction of body fat. However, the biggest problems of the chemotherapy are incomplete evidential materials and research findings on side effects. CLA is the abbreviation of conjugated linoleic acid, which was first sold as a diet food in the United States in 1998, and in Korea, was first approved by HK Biotech Co., Ltd. as an individually-approved health functional food product by the Korea Food & Drug Administration. Afterward, since additionally approved by several companies. CLA is more frequently sold on TV home shopping networks. While the effects of CLA in increasing body weight and inhibiting fat accumulation are clearly shown in animal studies, as side effects, insulin resistance and fatty liver are exhibited. The liver recognizes increased insulin and induces glucose breakdown by stopping glucose production, and when insulin resistance increases, such an action does not occur, thereby inducing type 2 diabetes. In clinical trials, a person with abdominal obesity showed the decrease in body weight and body fat after taking CLA, but side effects such as increased insulin resistance, high blood sugar and decreased HDL-cholesterol were exhibited. Recent study results showed that CLA can also induce acute hepatitis, and has hepatotoxicity that damages the liver.

Therefore, it has been demonstrated that CLA, which is a supplement for reducing body fat that is currently used around the world, has an effect in reducing body fat, but side effects causing hepatotoxicity are not addressed. Recently, as the obese population increases and the attention on diet increases, various dietary foods are developed, and due to the rapidly growing diet-related market, addressing the side effects of diet products is a very urgent task.

β-glucans are polysaccharides produced by polymerizing glucose with β-1,3 bonds, and include microorganism-derived β-glucans (β-1,3-glucan or β-1,3-1,6-glucan) produced by being isolated from polysaccharides of the cell walls or cells of microorganisms such as mushrooms, yeasts, etc., and vegetable β-glucan (β-1,3-1,4-glucan) produced by being extracted from cereals such as barley, oats, etc. These glucans may exhibit various physiological activities according to a more specific glucose bond structure, and are high value-added biomaterials used in various ways such as cosmetic additives, dietary supplements, food additives, concrete admixtures and feed additives.

Particularly, β-glucan, for example, in the form of β-(1,6)-branched (1,3)-glucan, known to be present in Schizophyllan of Schizophyllum commune has been reported to serve as a natural immune modulator that boosts immunity and does not have resistance and to have physiological activities for anticancer actions and antioxidation. β-glucans are well known as biological response modifiers (BRMs) that act on the human immune system to boost the immunity of the human body, and particularly, it has been reported that β-glucan enhances the overall functions of the immune system by activating the function of macrophages in the immune system, secreting cytokines such as interferons or interleukins, which are proliferation factors of other lymphocytes or leukocytes. In addition, it was confirmed that β-glucan inhibits necrosis to protect the liver despite liver damage caused by Taxol, which is an antitumor agent, and it has been reported that water-soluble β-glucan isolated from Agrobacterium sp. ZX09 also protects the liver from liver damage caused by alcohol. It was confirmed that β-glucan can protect the liver from the risk of liver damage.

### SUMMARY OF THE INVENTION

The present invention is directed to providing a pharmaceutical composition for preventing or treating obesity, which includes a supplement for reducing body fat and β-glucan as active ingredients, to suppress side effects caused by the supplement for reducing body fat.

However, technical problems to be solved in the present invention are not limited to the above-described problems, and other problems which are not described herein will be fully understood by those of ordinary skill in the art from the following descriptions.

The present invention provides a pharmaceutical composition for preventing or treating obesity, which includes a supplement for reducing body fat and β-glucan as active ingredients.

The composition may be for suppressing side effects caused by the supplement for reducing body fat.

The side effects may be due to hepatotoxicity or an increase in blood cholesterol level.

The supplement for reducing body fat may be at least one selected from CLA, HCA and dietary fiber, and preferably, CLA.

The β-glucan may be isolated from Schizophyllum commune or a culture thereof.

The β-glucan may have a structure of β-(1,6)-branched (1,3)-glucan.

The supplement for reducing body fat and the β-glucan may be mixed in a ratio of 2:1 (w/v:w/v) to 100:1 (w/v:w/v).

According to an exemplary embodiment of the present invention, a health functional food for preventing or alleviating obesity, which includes a supplement for reducing body fat and β-glucan as active ingredients, is provided.

According to another exemplary embodiment of the present invention, the use of a supplement for reducing body fat and β-glucan in a pharmaceutical composition for preventing or treating obesity is provided.

According to still another exemplary embodiment of the present invention, the use of a supplement for reducing body fat and β-glucan in a health functional food for preventing or alleviating obesity is provided.

According to yet another exemplary embodiment of the present invention, a method of treating obesity, which includes administering a supplement for reducing body fat and β-glucan to a subject, is provided.

The present invention relates to a composition for preventing or treating obesity, which includes a supplement for reducing body fat and β-glucan as active ingredients. The composition has advantages of a synergistic effect of the supplement for reducing body fat, and suppression of side effects caused by the supplement for reducing body fat, for example, hepatotoxicity and an increase in blood cholesterol level.

Therefore, the composition according to the present invention is expected to reduce body fat in a biocompatible way, and to be useful for prevention or treatment of obesity.

### BRIEF DESCRIPTION OF THE DRAWINGS

FIG. 1 is an image of comparing mice after 28 weeks of administering a test drug to Experimental Groups 1-1 to 1-2, and Comparative Experimental Groups 1-1 to 1-3.

FIG. 2 is a set of graphs of comparing body weights of mice, which are measured every week for 28 weeks after the start of administration of a test drug to Experimental Group 1-1 to 1-2, and Comparative Experimental Groups 1-1 to 1-3.

FIG. 3 is a table of comparing body weights and tissue weights of mice which have been sacrificed after 28 weeks of administering a test drug to Experimental Groups 1-1 to 1-2, and Comparative Experimental Groups 1-1 to 1-3.

FIG. 4 is a table of comparing serum analysis for mice which have been sacrificed after 28 weeks of administering a test drug to Experimental Group 1-1 to 1-2, and Comparative Experimental Groups 1-1 to 1-3.

FIG. 5 is a set of graphs of comparing body weights of mice, which are measured every week for 20 weeks after the start of administration of a test drug to Experimental Group 2-1 to 2-2, and Comparative Experimental Groups 2-1 to 2-3.

FIG. 6 is a table of comparing body weights and tissue weights of mice which have been sacrificed after 20 weeks of administering a test drug to Experimental Groups 2-1 to 2-2, and Comparative Experimental Groups 2-1 to 2-3.

FIG. 7 is a table of comparing serum analysis for mice which have been sacrificed after 20 weeks of administering a test drug to Experimental Group 2-1 to 2-2, and Comparative Experimental Groups 2-1 to 2-3.

### DETAILED DESCRIPTION OF THE INVENTION

The inventors had tried to overcome side effects caused by a supplement for reducing body fat, and thus confirmed that, when Schizophyllum commune-derived β-glucan was co-administered with the supplement for reducing body fat, compared to when the supplement for reducing body fat was administered alone, a weight loss effect may be enhanced, hepatotoxicity may be suppressed, and an increase in blood cholesterol content may be suppressed, and therefore, the present invention was completed. On the other hand, when Schizophyllum commune-derived β-glucan is administered alone, it is confirmed that it does not exhibit a significant weight loss effect.

Hereinafter, the present invention will be described in detail.

Pharmaceutical Composition for Preventing or Treating Obesity

The present invention provides a pharmaceutical composition for preventing or treating obesity, which includes a supplement for reducing body fat and β-glucan as active ingredients.

The pharmaceutical composition for preventing or treating obesity according to the present invention includes a supplement for reducing body fat and β-glucan as active ingredients.

The composition is for preventing or treating obesity, and such an effect may be confirmed by measuring a body weight and a tissue weight, or analyzing a triglyceride content and a free fatty acid content.

In addition, the composition may be for suppressing side effects caused by a supplement for reducing body fat, and specifically, the side effects may be due to hepatotoxicity or an increase in blood cholesterol level. More specifically, the hepatotoxicity may be confirmed by the measurement of a liver weight or the analysis of GOT/AST levels and γ-GPT/ALT levels.

The supplement for reducing body fat may be at least one selected from the group consisting from CLA. HCA and dietary fiber, and preferably, CLA or HCA. When the CLA or Garcinia cambogia extract is administered alone, a weight loss effect caused by reduced body fat is exhibited, but side effects caused by hepatotoxicity and an increase in blood cholesterol level are problematic.

Meanwhile, the β-glucan is administered in combination to suppress side effects caused by the supplement for reducing body fat, and preferably has the structure of β-(1.6)-branched (1,3)-glucan, but the present invention is not limited thereto. The β-glucan may be derived from microbial cells, yeast cells or mushroom mycelia, and more specifically, isolated from Schizophyllum commune or a culture thereof, but the present invention is not limited thereto.

To isolate the β-glucan form Schizophyllum commune, Schizophyllum commune mycelia may be obtained from a culture obtained by liquid culture. More specifically, as disclosed in Korean Patent No. 10-0892355 or 10-0909857, Schizophyllum commune was cultured, and β-glucan is preferably isolated and obtained from the culture thereof, but the present invention is not limited thereto.

When administered in combination by mixing a first solution containing the supplement for reducing body fat and a second solution containing the β-glucan, the supplement for reducing body fat and the β-glucan are preferably mixed in a ratio of 2:1 (w/v:w/v) to 100:1 (w/v:w/v), and the supplement for reducing body fat and the β-glucan are more preferably mixed in a ratio of 2:1 (w/v:w/v) to 10:1 (w/v:w/v), but the present invention is not limited thereto. Here, when the concentration of the supplement for reducing body fat is too small, the body weight reducing effect is insignificant, and when the concentration of the β-glucan is too small, hepatotoxicity or an increase in total cholesterol content in blood is not effectively suppressed.

Meanwhile, as the supplement for reducing body fat and the β-glucan are mixed in a ratio of 2:1 (w/v:w/v) to 100:1 (w/v:w/v), when the concentration of the β-glucan increases, hepatotoxicity and an increase in total cholesterol content in blood may be more effectively suppressed.

The pharmaceutical composition according to the present invention may be used in the formulation of an oral preparation such as powder, granules, tablets, capsules, suspensions, emulsions or syrups, a preparation for external use, a suppository and a sterile injectable solution according to a conventional method, and for formulation, suitable carriers, excipients or diluents conventionally used in the preparation of a pharmaceutical composition may be included.

As the carriers, excipients or diluents, various compound or mixtures including lactose, dextrose, sucrose, sorbitol, mannitol, xylitol, erythritol, maltitol, starch, acacia rubber, alginate, gelatin, calcium phosphate, calcium silicate, cellulose, methyl cellulose, microcrystalline cellulose, polyvinylpyrrolidone, water, methyl hydroxybenzoate, propyl hydroxybenzoate, talc, magnesium stearate and mineral oil may be used.

In preparation, generally used diluents or excipients such as a filler, a thickening agent, a binder, a wetting agent, a disintegrating agent and a surfactant may be used.

Solid preparations for oral administration may be prepared by mixing at least one excipient selected from starch, calcium carbonate, sucrose, lactose and gelatin with the supplement for reducing body fat and the β-glucan. In addition to simple excipients, lubricants such as magnesium stearate and talc may also be used.

Liquid preparations for oral administration may be suspensions, liquids for internal use, emulsions or syrups, and may include, in addition to simple diluents frequently used, various excipients, for example, a wetting agent, a sweetening agent, a flavoring agent, a preservative, etc.

Preparations for parenteral administration include a sterile aqueous solution, water, a non-aqueous vehicle, a suspending agent, an emulsifier, a lyophilizing agent, and a suppository. As a non-aqueous vehicle or a suspending agent, propylene glycol, polyethylene glycol, a vegetable oil such as olive oil, or an injectable ester such as ethyl oleate may be used. As a base material for suppositories, Witepsol®. Macrogol, Tween 61, cacao butter, laurin butter or glycerol gelatin may be used.

A preferred dose of the pharmaceutical composition according to the present invention may be determined according to a patient's condition, body weight, the severity of a disease, a drug type, an administration route and an administration duration, and properly selected by one of ordinary skill in the art. However, for a preferred effect, the pharmaceutical composition is preferably administered at 0.0001 μg/kg to 400 mg/kg, and more preferably, 0.001 to 200 mg/kg per day. The pharmaceutical composition may be administered once a day or several times by dividing the daily dose. However, the above-mentioned dose does not limit the scope of the present invention.

The pharmaceutical composition according to the present invention may be administered to mammals such as rats, mice, livestock or humans via various routes. All modes of administration, for example, oral, parenteral, rectal, intravenous, intramuscular, subcutaneous, intrauterine, and intracerebroventricular injections may be administered.

Health Functional Food for Preventing or Alleviating Obesity

In addition, the present invention provides a health functional food for preventing or alleviating obesity, which includes a supplement for reducing body fat and β-glucan as active ingredients.

The health functional food for preventing or alleviating obesity according to the present invention contains a supplement for reducing body fat and β-glucan as active ingredients, and the supplement for reducing body fat and the β-glucan are the same as described above.

In the health functional food for preventing or alleviating obesity according to the present invention, when the supplement for reducing body fat and the β-glucan are used as additives for the health functional food, they may be added by themselves or used with other food or food ingredients, and may be properly used according to a conventional method. The mixed amount of the active ingredients may be properly determined according to the purpose of use, for example, prevention, health or treatment.

The health functional food may be used in any formulation such as a common food or beverage form as well as powder, granules, pills, tablets or capsules.

The type of food is not particularly limited, and examples of food to which the material is added may be meat, sausages, bread, chocolate, candies, snacks, confectionery, pizza, ramen, other noodles, gum, dairy product including ice creams, various types of soup, beverages, tea, drinks, alcoholic beverages and vitamin complexes, and in the conventional sense, all types of food may be included.

Generally, in manufacture of food or beverages, the supplement for reducing body fat and the β-glucan may be added at 15 parts by weight or less, and preferably, 10 parts by weight or less with respect to 100 parts by weight of raw ingredient(s). However, in the case of long-term ingestion for health and hygiene or health control, the amount may be lower than the above-mentioned range. In addition, since the present invention has no problem in safety due to the use of a natural substance, the amount may be higher than the above-mentioned range.

A beverage of the health functional food according to the present invention may contain various flavoring agents or natural carbohydrates as additional ingredients, like conventional beverages. The above-described natural carbohydrates may include monosaccharides such as glucose and fructose, disaccharides such as maltose and sucrose, polysaccharides such as dextrin and cyclodextrin, and sugar alcohols such as xylitol, sorbitol and erythritol. Sweetening agents may include natural sweetening agents such as thaumatin and stevia extracts, and synthetic sweetening agents such as saccharin and aspartame. The proportion of the natural carbohydrate may be approximately 0.01 to 0.04 g. and preferably, approximately 0.02 to 0.03 g per 100 mL of the beverage according to the present invention.

In addition to the above ingredients, the health functional food for preventing or alleviating obesity according to the present invention may contain various nutrients, vitamins, electrolytes, flavors, colorants, pectic acid and a salt thereof, alginic acid and a salt thereof, organic acids, protective colloidal thickening agents, pH regulators, stabilizers, preservatives, glycerin, and a carbonating agent used in an alcoholic or carbonated drink. In addition, the composition for preventing or alleviating obesity according to the present invention may contain pulp for the production of natural fruit juice, a fruit juice drink or a vegetable drink. These ingredients may be used independently or in combination. Generally, the proportion of the additive may be, but is not limited to, 0.01 to 0.1 part by weight with respect to 100 parts by weight of the health functional food of the present invention.

Further, the present invention provides the use of a supplement for reducing body fat and β-glucan in a pharmaceutical composition for preventing or treating obesity.

In addition, the present invention provides the use of a supplement for reducing body fat and β-glucan in a health functional food for preventing or alleviating obesity.

In addition, the present invention provides a method of treating obesity, which includes administering a supplement for reducing body fat and β-glucan to a subject.

The term “subject” used herein refers to a target in need of treatment, and more specifically, a mammal such as a human or a non-human primate, a mouse, a rat, a dog, a cat, a horse, or a cow.

As described above, the present invention relates to a composition for preventing or treating obesity, which includes a supplement for reducing body fat and β-glucan as active ingredients, and the composition has advantages of a synergistic effect of the supplement for reducing body fat, and suppression of side effects caused by the supplement for reducing body fat, for example, hepatotoxicity and an increase in blood cholesterol level.

Therefore, the composition according to the present invention is expected to reduce body fat in a biocompatible way, and to be useful for prevention or treatment of obesity.

### EXAMPLES

Hereinafter, to help in understanding the present invention, exemplary examples will be suggested. However, the following examples are merely provided to more easily understand the present invention, and not to limit the present invention.

### Example 1: Confirmation of Synergistic Effect of Supplement for Reducing Body Fat and Effect of Suppressing Side Effects Due to Co-Administration of CLA with β-Glucan (Animal Test)

Six-week-old wild-type C57BL/6 mice, to be subjected to body fat monitoring, were purchased, acclimated for 3 days and then raised. Specifically, the mice were divided into a total of five groups of 6 mice, as shown in Table 1, CLA (Sigma-Aldrich). Schizophyllum commune-derived β-glucan (SPG with the structure of β-(1,6)-branched (1,3)-glucan; Quegen Biotech Co., Ltd.) and a phosphate buffer solution (PBS) were orally administered as test drugs in combination or independently once every day. Specifically, 0.2 mL of CLA (concentration=10 mg/mL), and after 30 minutes, 0.2 mL of Schizophyllum commnune-derived β-glucan (SPG with the structure of β-(1,6)-branched (1,3)-glucan; concentration=0.4 mg/mL) were orally administered to Experimental Group 1-1. In addition, 0.2 mL of CLA (concentration=10 mg/mL), and after 30 minutes, 0.2 mL of Schizophyllum commune-derived β-glucan (SPG with the structure of β-(1,6)-branched (1,3)-glucan; concentration=2 mg/mL) were orally administered to Experimental Group 1-2. Meanwhile, 0.2 mL of CLA (concentration=10 mg/mL) was orally administered to Comparative Experimental Group 1-1. In addition, 0.2 mL of Schizophyllum commune-derived β-glucan (SPG with the structure of β-(1,6)-branched (1,3)-glucan; concentration=2 mg/mL) was orally administered to Comparative Experimental Group 1-2, and 0.2 mL of PBS was orally administered to Comparative Experimental Group 1-3.

TABLE 1

| | | | Number of | Total |
|---|---|---|---|---|
| | | | adminis- | adminis- |
| | Concentration of | | trations | tration |
| | administered drug | Dose | per week | period |
| Experimental | CLA 10 mg/mL + | 0.2 mL each | 5 times | 28 weeks |
| Group 1-1 | SPG 0.4 mg/ml | | | |
| Experimental | CLA 10 mg/mL + | 0.2 mL each | 5 times | 28 weeks |
| Group 1-2 | SPG 2 mg/ml | | | |
| Comparative | CLA 10 mg/mL | 0.2 mL | 5 times | 28 weeks |
| Experimental | | | | |
| Group 1-1 | | | | |
| Comparative | SPG 2 mg/mL | 0.2 mL | 5 times | 28 weeks |
| Experimental | | | | |
| Group 1-2 | | | | |
| Comparative | PBS | 0.2 mL | 5 times | 28 weeks |
| Experimental | | | | |
| Group 1-3 | | | | |

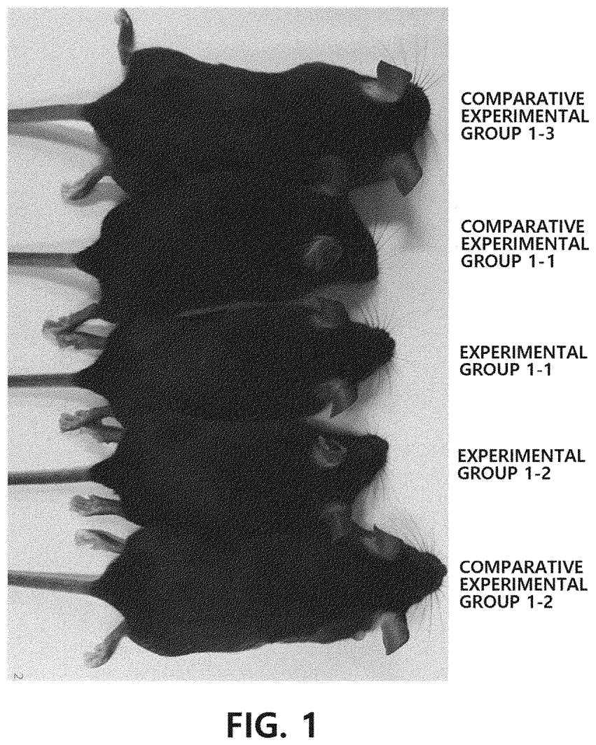

The final result of the experimental animals after 28 weeks for each administered group is shown in FIG. 1. Here, in animal models with increased body fat, as the supplement for reducing body fat, that is, CLA, and β-glucan were orally administered in combination, it was confirmed whether the synergistic effect and the effect of suppressing side effects of the supplements for reducing body fat are exhibited.

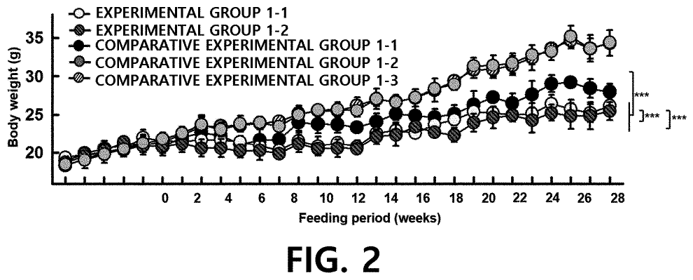

Specifically, in Experimental Groups 1-1 and 1-2 and Comparative Experimental Groups 1-1 to 1-3, the body weights of the mice were measured every week for 28 weeks after the start of administration of the test drugs, and the result is shown in FIG. 2.

As shown in FIG. 2, it is confirmed that, compared to Comparative Experimental Group 1-3, Comparative Experimental Group 1-1 was greatly decreased in body weight, but Comparative Experimental Group 1-2 had no change in body weight, as the period of administering the test drugs progressed. Meanwhile, it is confirmed that, compared to Comparative Experimental Group 1-1 administered CLA alone, Experimental Groups 1-1 and 1-2 co-administered CLA and SPG were further decreased in body weight as the period of administering the test drugs progressed. That is, the synergistic effect of the supplement for reducing body fat (CLA) was confirmed. Particularly, it is confirmed that, compared to Experimental Group 1-1, Experimental Group 1-2 was greatly decreased in body weight as the period of administering the test drugs progressed.

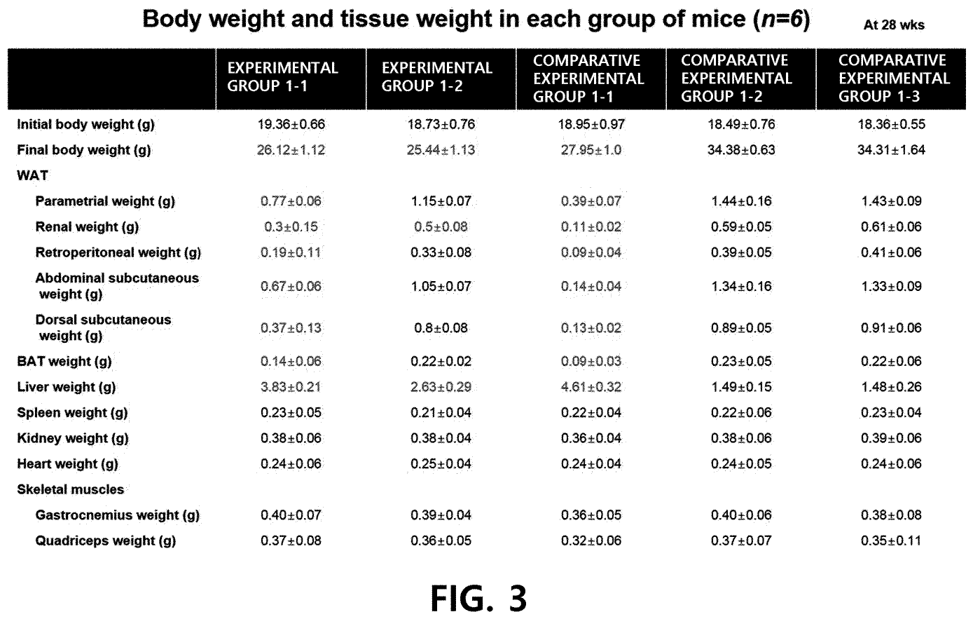

In addition, in Experimental Groups 1-1 and 1-2 and Comparative Experimental Groups 1-1 to 1-3, the mice were sacrificed after 28 weeks of administering the test drugs, and then body weights and tissue weights thereof measured, and the result is shown in FIG. 3.

As shown in FIG. 3, it is confirmed that, compared to Comparative Experimental Group 1-3, Experimental Groups 1-1 and 1-2 and Comparative Experimental Group 1-1 orally administered the supplement for reducing body fat, that is. CLA, were greatly decreased in body weight and tissue weight of each organ. However, it is confirmed that Comparative Experimental Group 1-1 was rather increased in tissue weight of the liver. That is, it seems that the supplement for reducing body fat, that is, CLA, leads to side effects caused by hepatotoxicity. It is confirmed that, compared to Comparative Experimental Group 1-3. Comparative Experimental Group 1-2 had almost no change in body weight and tissue weight of each organ.

Meanwhile, it is confirmed that, compared to Comparative Experimental Group 1-1, Experimental Groups 1-1 and 1-2 were further decreased in body weight, and also decreased in tissue weight of the liver. In addition, it is confirmed that there was no significant difference in tissue weights of the spleen, kidney, heart and muscle.

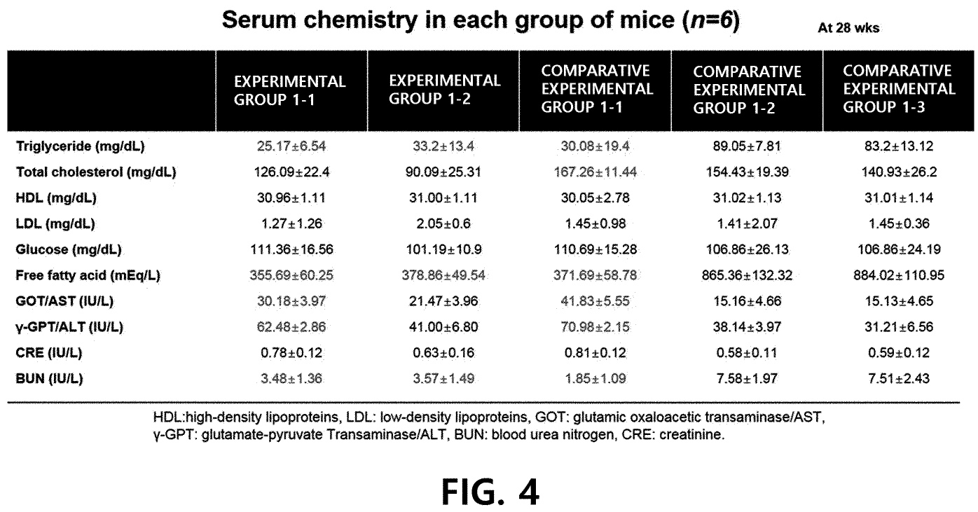

In addition, in Experimental Groups 1-1 and 1-2 and Comparative Experimental Groups 1-1 to 1-3, the mice were sacrificed after 28 weeks of administering the test drugs, and then subjected to serum analysis, and the result is shown in FIG. 4.

As shown in FIG. 4, it is confirmed that, compared to Comparative Experimental Group 1-3, in Experimental Groups 1-1 and 1-2 and Comparative Experimental Group 1-1 orally administered the supplement for reducing body fat, that is, CLA, triglyceride and free fatty acid contents were surprisingly decreased by more than half. However, it was confirmed that, compared to Comparative Experimental Group 1-3, Comparative Experimental Group 1-1 was increased in total cholesterol content in blood and had liver damage. It can be seen that the supplement for reducing body fat, that is. CLA, leads to an increase in total cholesterol content in blood, and as described in FIG. 3, side effects caused by hepatotoxicity (e.g., an increased liver weight).

Meanwhile, it can be confirmed that, compared to Comparative Experimental Group 1-1 or 1-3, in Experimental Groups 1-1 and 1-2 co-administered SPG, the total cholesterol content in blood was greatly reduced, and hepatotoxicity was minimized. Particularly, it was confirmed that, compared to Comparative Experimental Group 1-2, in Experimental Groups 1-1 and 1-2, a total cholesterol content in blood was greatly lowered, and the higher the β-glucan level, the lower the total cholesterol content in blood and hepatotoxicity.

### Example 2: Confirmation of Synergistic Effect of Supplement for Reducing Body Fat and Effect of Suppressing Side Effects Due to Co-Administration of Garcinia cambogia Extract (HCA) with β-Glucan (Animal Test)

Six-week-old wild-type C57BL/6 mice, to be subjected to body fat monitoring, were purchased, acclimated for 3 days and then raised. Specifically, the mice were divided into a total of five groups of 10 mice, as shown in Table 2. HCA (Sigma-Aldrich), Schizophyllum commune-derived β-glucan (SPG with the structure of β-(1.6)-branched (1,3)-glucan; Quegen Biotech Co., Ltd.) and a phosphate buffer solution (PBS) were orally administered as test drugs in combination or independently once every day. Specifically, 0.2 mL of HCA (concentration=10 mg/mL), and after 30 minutes, 0.2 mL of Schizophyllum commune-derived β-glucan (SPG with the structure of β-(1,6)-branched (1,3)-glucan; concentration=0.4 mg/mL) were orally administered to Experimental Group 2-1. In addition, 0.2 mL of HCA (concentration=10 mg/mL), and after 30 minutes, 0.2 mL of Schizophyllum commune-derived β-glucan (SPG with the structure of β-(1,6)-branched (1,3)-glucan; concentration=2 mg/mL) were orally administered to Experimental Group 2-2. Meanwhile, 0.2 mL of HCA (concentration=10 mg/mL) was orally administered to Comparative Experimental Group 2-1. In addition, 0.2 mL of Schizophyllum commune-derived β-glucan (SPG with the structure of 3-(1.6)-branched (1,3)-glucan:concentration=2 mg/mL) was orally administered to Comparative Experimental Group 2-2, and 0.2 mL of PBS was orally administered to Comparative Experimental Group 2-3.

TABLE 2

| | | | Number of | Total |
|---|---|---|---|---|
| | | | adminis- | adminis- |
| | Concentration of | | trations | tration |
| | administered drug | Dose | per week | period |
| Experimental | HCA 10 mg/mL + | 0.2 mL each | 5 times | 20 weeks |
| Group 2-1 | SPG 0.4 mg/mL | | | |
| Experimental | HCA 10 mg/mL | 0.2 mL each | 5 times | 20 weeks |
| Group 2-2 | SPG 2 mg/mL | | | |
| Comparative | HCA 10 mg/mL | 0.2 mL | 5 times | 20 weeks |
| Experimental | | | | |
| Group 2-1 | | | | |
| Comparative | SPG 2 mg/mL | 0.2 mL | 5 times | 20 weeks |
| Experimental | | | | |
| Group 2-2 | | | | |
| Comparative | PBS | 0.2 ml | 5 times | 20 weeks |
| Experimental | | | | |
| Group 2-3 | | | | |

Here, in animal models with increased body fat, as the supplement for reducing body fat, that is, HCA, and β-glucan were orally administered in combination, it was confirmed whether the synergistic effect and the effect of suppressing side effects of the supplements for reducing body fat are exhibited.

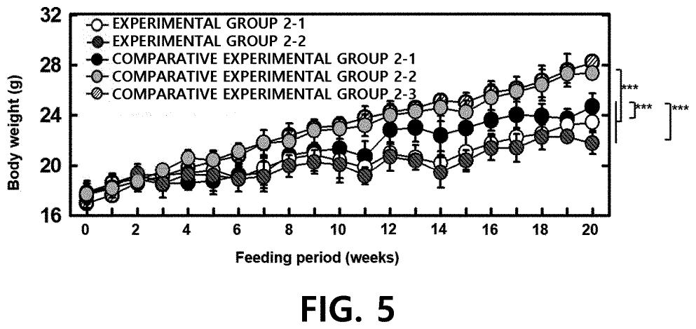

Specifically, in Experimental Groups 2-1 and 2-2 and Comparative Experimental Groups 2-1 to 2-3, the body weights of the mice were measured every week for 20 weeks after the start of administration of the test drugs, and the result is shown in FIG. 5.

As shown in FIG. 5, it is confirmed that, compared to Comparative Experimental Group 2-3, Comparative Experimental Group 2-1 was greatly decreased in body weight, but Comparative Experimental Group 2-2 had no change in body weight, as the period of administering the test drugs progressed. Meanwhile, it is confirmed that, compared to Comparative Experimental Group 2-1 administered HCA alone, Experimental Groups 2-1 and 2-2 co-administered HCA and SPG were further decreased in body weight as the period of administering the test drugs progressed. That is, the synergistic effect of the supplement for reducing body fat (HCA) was confirmed. Particularly, it is confirmed that, compared to Experimental Group 2-1. Experimental Group 2-2 was greatly decreased in body weight as the period of administering the test drugs progressed.

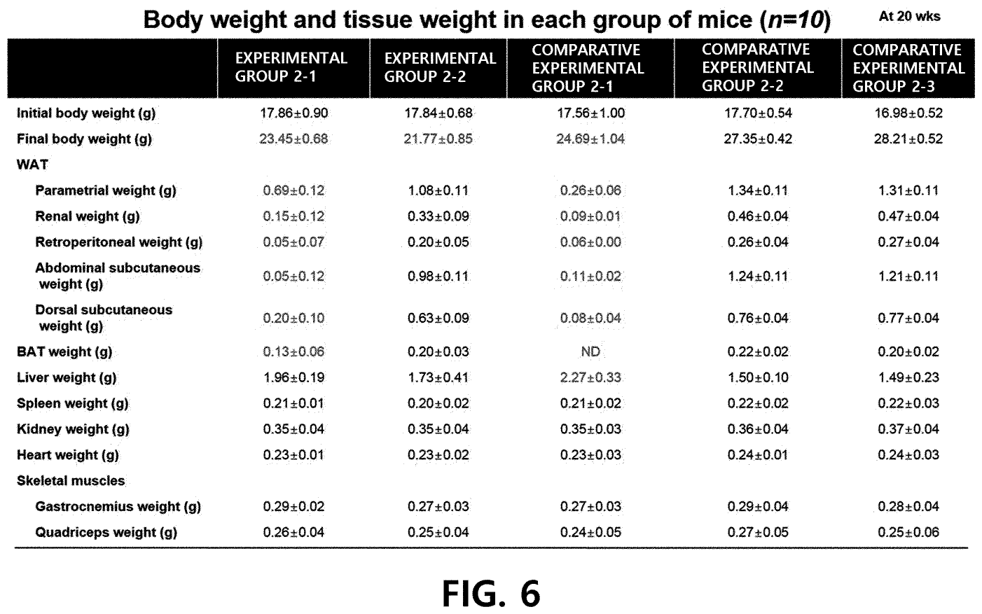

In addition, in Experimental Groups 2-1 and 2-2 and Comparative Experimental Groups 2-1 to 2-3, the mice were sacrificed after 20 weeks of administering the test drugs, and then body weights and tissue weights thereof measured, and the result is shown in FIG. 6.

As shown in FIG. 6, it is confirmed that, compared to Comparative Experimental Group 2-3, Experimental Groups 2-1 and 2-2 and Comparative Experimental Group 2-1 orally administered the supplement for reducing body fat, that is. HCA, were greatly decreased in body weight and tissue weight of each organ. However, it is confirmed that Comparative Experimental Group 2-1 was rather increased in tissue weight of the liver. That is, it seems that the supplement for reducing body fat, that is, HCA, leads to side effects caused by hepatotoxicity. It is confirmed that, compared to Comparative Experimental Group 2-3, Comparative Experimental Group 2-2 had almost no change in body weight and tissue weight of each organ.

Meanwhile, it is confirmed that, compared to Comparative Experimental Group 2-1. Experimental Groups 2-1 and 2-2 were further decreased in body weight, and also decreased in tissue weight of the liver. In addition, it is confirmed that there was no significant difference in tissue weights of the spleen, kidney, heart and muscle.

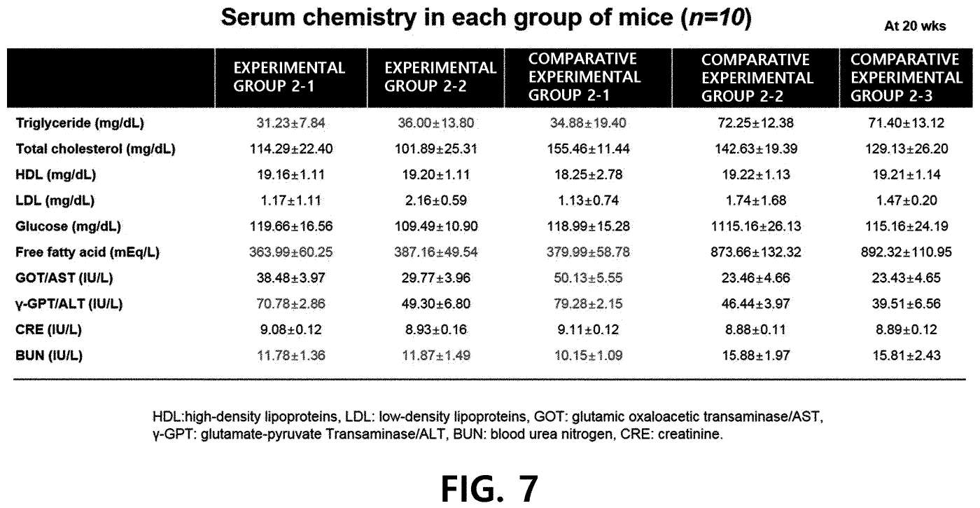

In addition, in Experimental Groups 2-1 and 2-2 and Comparative Experimental Groups 2-1 to 2-3, the mice were sacrificed after 20 weeks of administering the test drugs, and then subjected to serum analysis, and the result is shown in FIG. 7.

As shown in FIG. 7, it is confirmed that, compared to Comparative Experimental Group 2-3, in Experimental Groups 2-1 and 2-2 and Comparative Experimental Group 2-1 orally administered the supplement for reducing body fat, that is, HCA, triglyceride and free fatty acid contents were surprisingly decreased by more than half. However, it was confirmed that, compared to Comparative Experimental Group 2-3, Comparative Experimental Group 2-1 was increased in total cholesterol content in blood and had liver damage. It can be seen that the supplement for reducing body fat, that is. HCA, leads to an increase in total cholesterol content in blood, and as described in FIG. 6, side effects caused by hepatotoxicity (e.g., an increased liver weight).

Meanwhile, it can be confirmed that, compared to Comparative Experimental Group 2-1 or 2-3, in Experimental Groups 2-1 and 2-2 co-administered SPG, the total cholesterol content in blood was greatly reduced, and hepatotoxicity was minimized. Particularly, it was confirmed that, compared to Comparative Experimental Group 2-2, in Experimental Groups 2-1 and 2-2, a total cholesterol content in blood was greatly lowered, and the higher the β-glucan level, the lower the total cholesterol content in blood and hepatotoxicity.

Hereinafter, preparation examples for compositions containing the compound of the present invention will be described. The present invention is merely provided to describe the examples in detail, but not limited thereto.

### Preparation Example 1: Preparation of Powder

Supplement for reducing body fat, 400 mg and β-glucan, 20 mg

Lactose hydrate, 100 mg

Talc, 10 mg

A powder was prepared by mixing the ingredients and filling an airtight pouch with the mixture.

### Preparation Example 2: Preparation of Tablet

Supplement for reducing body fat, 100 mg and β-glucan, 20 mg

Corn starch, 100 mg

Lactose hydrate, 100 mg

Magnesium stearate, 2 mg

A tablet was prepared by mixing the ingredients and tableting the mixture according to a conventional method of preparing a tablet.

### Preparation Example 3: Preparation of Capsule

Supplement for reducing body fat, 100 mg and β-glucan, 20 mg

Microcrystalline cellulose, 3 mg

Lactose hydrate, 14.8 mg

Magnesium stearate, 0.2 mg

A capsule was prepared by mixing the ingredients and filling a gelatin capsule with the mixture according to a conventional method of preparing a capsule.

### Preparation Example 4: Preparation of Injection

Supplement for reducing body fat, 50 mg and β-glucan, 10 mg

Mannitol, 180 mg

Sterile distilled water for injection, 2974 mg

Sodium hydrogen phosphate, 26 mg

An injection was prepared by mixing the above-described ingredients to contain them per ampoule (2 mL) according to a conventional method of preparing an injection.

### Preparation Example 5: Preparation of Liquid

Supplement for reducing body fat, 100 mg and β-glucan, 10 mg

Isomerized glucose syrup, 10 g

Mannitol, 5 g

Distilled water, q. s.

Lemon flavor, q. s.

A liquid was prepared by dissolving the above-described ingredients in distilled water according to a conventional preparation method, adding an appropriate amount of lemon flavor, adding distilled water to adjust a total volume to 100 mL, and then filling a sterile brown bottle with the resulting mixture.

### Preparation Example 6: Preparation of Health Functional Food

Supplement for reducing body fat, 800 mg and β-glucan, 40 mg

Vitamin mixture, q.s.

Vitamin A acetate, 70 μg

Vitamin E, 1.0 mg

Vitamin B₁, 0.13 mg

Vitamin B₂, 0.15 mg

Vitamin B₆. 0.5 mg

Vitamin B₁₂, 0.2 μg

Vitamin C, 10 mg

Biotin, 10 μg

Nicotinic acid amide, 1.7 mg

Folic acid, 50 μg

Calcium pantothenate, 0.5 mg

Mineral mixture, q. s.

Ferrous sulfate, 1.75 mg

Zinc oxide, 0.82 mg

Magnesium carbonate, 25.3 mg

Potassium monophosphate, 15 mg

Potassium diphosphate, 55 mg

Potassium citrate, 90 mg

Calcium carbonate, 100 mg

Magnesium chloride, 24.8 mg

The mixture of vitamins and minerals was prepared by mixing relatively suitable ingredients for a health functional food in a preferred composition ratio, but the mixing ratio may be arbitrarily modified. A granule was prepared by mixing the above-described ingredients according to a conventional method of preparing a health functional food, and can be used in preparation of a health functional food according to a conventional method.

### Preparation Example 7: Preparation of Health Drink

Supplement for reducing body fat, 400 mg and β-glucan, 10 mg

Vitamin C, 15 g

Vitamin E (powder), 100 g

Iron lactate, 19.75 g

Zinc oxide, 3.5 g

Nicotinic acid amide, 3.5 g

Vitamin A, 0.2 g

Vitamin B₁, 0.25 g

Vitamin B₂, 0.3 g

Distilled water, q. s.

A health drink composition of the present invention was prepared by mixing the above-described ingredients according to a conventional method of preparing a health drink, heating the mixture while stirring for 1 hour at 85° C., filtering the resulting solution, putting the filtered solution into a sterilized 2 L container, followed by sealing and sterilization, and then storing the sterilized product in a refrigerator.

Although the composition was prepared by mixing ingredients relatively suitable for beverage preference, a mixing ratio may be arbitrarily modified according to a regional or ethnic preference such as a class of people or countries that consume the composition, or the purpose of use thereof.

It should be understood by those of ordinary skill in the art that the above descriptions of the present invention are exemplary, and the example embodiments disclosed herein can be easily modified into other specific forms without changing the technical spirit or essential features of the present invention. Therefore, it should be interpreted that the example embodiments described above are exemplary in all aspects, and are not limitative.

1. A method for preventing or treating obesity, the method comprising administering a supplement for reducing body fat and β-glucan to a subject in need thereof.

2. The method of claim 1, which is for suppressing side effects caused by the supplement for reducing body fat.

3. The method of claim 2, wherein the side effects are caused by hepatotoxicity or an increase in total cholesterol content in blood.

4. The method of claim 1, wherein the supplement for reducing body fat is one or more selected from the group consisting of conjugated linoleic acid, a Garcinia cambogia extract and dietary fiber.

5. The method of claim 4, wherein the supplement for reducing body fat is conjugated linoleic acid or a Garcinia cambogia extract.

6. The method of claim 1, wherein the β-glucan is isolated from Schizophyllum commune or a culture thereof.

7. The method of claim 1, wherein the β-glucan has a structure of β-(1,6)-branched (1,3)-glucan.

8. The method of claim 1, wherein the supplement for reducing body fat and the β-glucan are mixed in a ratio of 2:1 (w/v:w/v) to 100:1 (w/v:w/v).

9-12. (canceled)
